(19) United States
(12) Patent Application Publication
(10) Pub. No.: US 2013/0217869 A1
(43) Pub. Date: Aug. 22, 2013

(54) METHOD FOR PRODUCTION OF LIGNIN

(71) Applicants: Thomas Ters, Wien (AT); Halim Fuqaha, Tulln (AT); Anton Friedl, Wien (AT); Ewald Srebotnik, Wien (AT); Florian Zikeli, Neusiedl am See (AT); Kurt Messner, Wien (AT)

(72) Inventors: Thomas Ters, Wien (AT); Halim Fuqaha, Tulln (AT); Anton Friedl, Wien (AT); Ewald Srebotnik, Wien (AT); Florian Zikeli, Neusiedl am See (AT); Kurt Messner, Wien (AT)

(73) Assignee: ANNIKKI GMBH, Graz (AT)

(21) Appl. No.: 13/882,485

(22) PCT Filed: Oct. 28, 2011
(86) PCT No.: PCT/AT11/00438

§ 371 Date: Apr. 29, 2013

(30) Foreign Application Priority Data
Oct. 29, 2010 (AT) A 1799/2010

(51) Int. Cl. C07G 1/00; C07C 51/47

(52) U.S. Cl. CPC C07G 1/00; C07C 51/47
USPC 530/507; 530/500; 562/478

(57) ABSTRACT

A method for the preparation of lignin, characterized in that an acid is added to an alkaline alcoholic solution of lignin and the precipitated lignin is separated, and, optionally, from the filtrate alcohol is removed in order to get further lignin; as well as a method for the preparation of a concentrated alkaline, alcoholic solution of lignin by membrane filtration; the use of the lignin thus prepared; and the isolation of hydroxy cinnamic acids from the solution, from which the lignin has been separated.

In response to dwindling oil reserves and possible uncertainties with oil supply, biorefinery processes have been gaining great importance. This is the decomposition, of biomass into its individual components and the further processing thereof into higher-value products. The most economic raw material is lignocellulose, like wood or annual plants (e.g. straw of cereals), which are available in large quantities and which do not compete with food stuff. An essential economic factor in the use thereof is the so-called pulping process (“pre-treatment”). An ideal pulping method decomposes lignocellulose without significant energy consumption as selectively as possible into its main components cellulose, hemicellulose and lignin. In particular lignin has developed to constitute a desirable starting material for chemical industry as a possible replacement for petrochemical materials and has the prospects for use in low-molecular form, for example as fuel components, adhesives, binders, or in higher-molecular form as polymer replacement or polymer additive, resin components, carbon fibres, etc. The type of the pulping process will substantially influence thereby the properties of the lignin prepared as well as the downstream processing of the lignin fraction. The majority of the pulping processes is carried out at temperatures between 100° and 200° C., contains thermal, degradation and conversion products of cellulose, hemicellulose and lignin, which are then separated by way of distillation under high energy consumption, such as, e.g. in organosolv processes. (C. Arato, E. K. Pye, G. Gjennestad, 2005, The Lignol approach to biorefining of woody biomass to produce ethanol and chemicals. Appl. Biochem. Biotechnol., Vol. 121-12:871-882, Pilot project “Lignocellulose-Bioraffinerie” Geinsamer Sclussbericht zu den wissenschafilichen Ergebnissen aller Teihorhaben).

Lignin is an important raw material, which may be used, because of its structure, as a biomaterial, for example, as a component, in thermosetting plastics (“Duroplaste”), or in plastics, or also for the preparation of chemical products and in biorefinery, and which may be prepared, for example, from biomass, e.g. following the alkaline pulping process thereof. Lignin precipitation from alkaline solutions prepared thereby by acidification is known. Lignin from the alkaline pulping upon acid precipitation, however, cannot be filtered in general anymore but first has to be subjected before to an ageing process, consisting of heating and letting rest. If, for example, lignocellulose is pulped by the soda process without the addition of a solvent, (e.g. J. H. Lora & E. Escudero, 2000, Soda pulping of agricultural fibres for boardmaking applications. Paper Technology, May 2000, 37-42.), the lignin obtained cannot be filtered. For its separation and preparation, there is suggested subsequent heating and ageing (see A. Abaecherli, F. Doppenberg, 2003, Method for preparing alkaline solutions containing aromatic polymers, EP0970275).

Surprisingly, now it has been found that lignin may be separated by filtration, if lignocellulose has been pulped by an alkaline, alcoholic solution below 100° C. and the lignin is subjected to acid precipitation from this alkaline, alcoholic solution, whereby a preceding concentration step may be carried out, for example a preceding separation of alcohol from the solution.

In one aspect the present invention provides a method for the preparation of lignin, characterized in that an acid is added to an alkaline alcoholic solution of lignin and the precipitated lignin is separated, and, optionally, from the filtrate alcohol is removed in order to prepare further lignin.

By addition of an acid in a method according to the present invention, the pH of the alcoholic, alkaline, lignin-containing solution, e.g. pulping solution, is decreased by the addition of an acid to a pH of pH 7 to pH 1, preferably from pH 7 to pH 2, especially preferably to pH 2.5 to 1.5.

In a further aspect the pH value of the alcoholic, alkaline solution is decreased by addition of an acid step-by-step between pH 7 and pH 1, wherein the precipitated lignin is separated, in particular separated by centrifugation, filtration, ultra-filtration, following each step of the pH decrease, in particular wherein the lignin fractions obtained are dried and the alcohol possibly still present in the lignin fraction/s obtained is recovered by drying.

A method for the preparation of lignin, which is provided according to the present invention herein is designated also as “method according to (of) the present invention”.

Surprisingly, it has been shown that, according to a method according to the present invention, lignin tractions with higher molecular weight are precipitated by acid addition, while lignin fraction of lower molecular weight compared to the precipitated fractions remain in solution. Upon removal of the precipitated lignin fractions, e.g. by filtration, further lignin fractions may be precipitated by the removal of alcohol from the remaining solution, e.g. from the remaining filtrate. A method according to the present invention, hence, is especially useful for the preparation of lignin in fractionated form, i.e. the preparation of lignin fractions of various molecular weights.

In a further aspect the present invention provides a method for the fractionated preparation of higher- and low-molecular lignin, characterized in that an acid is added to an alkaline, alcoholic solution of lignin, the precipitated higher-molecular lignin fraction is separated, and, from the solution obtained upon separation, alcohol is removed, preferably by vaporization, vacuum vaporization, distillation, vacuum distillation, in order to precipitate low-molecular lignin fractions.

In a method according to the present invention the low-molecular lignin traction has an Mn (number average molecular weight) of 1000 D to 650 D, preferably 750 D, and an Mw (weight average molecular weight) of 1500 D to 1000 D, preferably 1250 D. The high-molecular lignin fraction has an Mn of 1500 D to 1000 D, preferably 1100 D to 1200 D, and an Mw of 7000 D to 3000 D, preferably 4500 D 5000 D.

Average molecular weight herein is intended to indicate the average weight (Mw) and/or average number (Mn) of the molecular weight distribution, calculated under use of the Agilent-Chemstation-GPC Data Analysis Software, wherein the molecular weight distribution is determined chromatographically by HPLC using three TSK-GEL-columns (Tosoh G4000PW/G3000PW/G3000PW) coupled ion series, 10 mM sodium hydroxide as mobile phase, UV detection at 280 nm as well as PSS (poly(styrol sulfonate) sodium salt) calibration standards in die molecular weight range from 0.3 kD to 35 kD.

An alkaline, alcoholic solution of lignin may be prepared, for example, by treating lignocellulosic material with an aqueous solution containing an alcohol, in particular a C₁₋₆ alcohol, and having a pH between 11.0 and 14.0, thereby cleaving lignocellulose and thereby providing and separating a material enriched with cellulose and hemicellulose. Surprisingly, lignin fractions remain practically quantitatively in solution.

As an alcohol preferably a water-soluble alcohol is used, e.g. an aliphatic alcohol, such as a C₁₋₆ alcohol, or mixtures of such alcohols, in particular preferably a C₁₋₄ alcohol, such as ethanol, isopropanol. In a preferred embodiment there is used ethanol, in another preferred embodiment there is used isopropanol as an alcohol.

Alcohol is present in an aqueous solution in the pulping process and in the method according to the invention preferably in a range from 10 to 70% (vol/vol), e.g. from 30 to 60% (vol/vol), it was found that, in order to achieve high selectivity of the lignin degradation, about 30% (vol/vol), and, in order to achieve a high lignin yield, about 60% (vol/vol) may be used.

The lignocellulose (pulping) is cleaved at a temperature below 100° C., such as 80° C., e.g. below 60° C., preferably from 40° C. to 90° C., especially preferably from 50° C. to 70° C.

The pulping period is preferably between 2 hours to 36 hours, especially preferably 3 to 18 hours.

For cleavage of the lignocellulosic material, the lignocellulosic material is present in the aqueous, alkaline, alcoholic mixture, preferably in a material density of 3-40% by weight, such as 5-40% by weight, in particular 5-20% by weight.

The pH of the aqueous mixture between 11.0 and 14.0 for the cleavage may be adjusted by use of a base, preferably an inorganic base, such as an inorganic hydroxide, in particular a hydroxide of an alkali or earth alkali metal, e.g. potassium hydroxide, sodium hydroxide, preferably sodium hydroxide.

The pulping solution is composed of a base, an alcohol, in particular a C₁₋₄ alcohol and water. The concentration of the base based upon the dry weight of the lignocellulose to be pulped is 3 to 12% by weight, dependent on the lignification level of the substrate, in particular preferably 5 to 8% by weight.

For pulping shredded lignocellulose, preferably shredded straw, is mixed in a reaction vessel with the pre-heated reaction solution. The solid content may be at the beginning of the pulping process between 3-40% by weight, usually, however, between 5 and 20% by weight, e.g. 10-20% by weight.

As a lignocellulosic material preferably straw, energy crops such as switch grass, elephant grass or abaca, sisal, bagasse, or untypical lignocellulose substrates like glumes, such as rice husks, preferably straw, energy crops, bagasse or glumes, especially preferably straw or bagasse, e.g. straw, is used.

Straw has a strongly hydrophobic surface, so that wetting with aqueous solutions is a problem. St has been shown that, by use of alcohol, it is possible to introduce the reaction solution into the pores of the substrate even without pressure, and to replace the air present by the reaction solution. Furthermore, it has been shown that under the selected reaction conditions alcohol accelerates the extraction of the cleavage products from straw and that it contributes to maintaining the lignin cleavage products in solution and that, in addition, alcohol decreases the solubility of the hemicellulose and the cleavage products thereof and, hence, the hemicellulose is maintained in the substrate.

Depending on the further intended use of the products, by combination of the parameters NaOH concentration, temperature, duration, material density and alcohol concentration, there may be achieved either a maximal lignin yield of about 90% with little hemicellulose degradation (about 10%), or a lignin yield of about 70% with less than 1% hemicellulose degradation. The use of alcohol, hence, allows for a comprehensive decoupling of the lignin cleavage from the hemicellulose cleavage.

By appropriate selection of the concentration of the pulping components alcohol, water and base as well as the reaction parameters temperature, pulping duration and solid concentration for the respective lignocellulose to be pulped, the desired lignin yield may be adjusted. E.g. regression models for pulping data for the respective lignocellulose to be pulped may be established, from which the optimum composition of the pulping components in percent and the optimum reaction parameters may be selected for each desired lignin content of the solid obtained upon pulping. The establishment of such regression models may be realized by statistical experimental planning.

For the case that the method according to the invention is predominantly intended as a biorefinery process, the lignin which is dissolved during the pulping step is separated from the solid. This may be realized, for example, by filtration, centrifugation or pressing, e.g. with the help of a screw press. The solid is subjected further to a washing process.

The lignin-containing pulping solution is separated from the solid, for example, by filtration or centrifugation.

In another aspect the present invention provides a method for the preparation of lignin from a lignocellulosic material by pulping with alcohol, in particular a C₁₋₄ alcohol, in particular ethanol; water and a base, in particular a sodium hydroxide, at a temperature below 100° C., in particular from 40° C. to 90° C., in particular from 50° C. to 70° C., in particular such, that the concentration of the base based on the dry weight of the lignocellulose to be pulped is from 3 to 12% by weight, in particular from 4 to 10% by weight, in particular from 5 to 8% by weight, characterized in that the lignin present in the alkaline, alcoholic solution is separated from the solid, in particular separated by pressing, centrifugation, filtration, and the lignin is separated from the pulping solution by addition of an acid in the form of a soluble lower-molecular and an insoluble higher-molecular lignin fraction, in particular wherein the higher-molecular fraction is precipitated by addition of the acid and is subsequently separated, and the low-molecular fraction is precipitated by removal of the alcohol from the solution and is subsequently separated.

The preparation of the lignin from the pulping solution following the separation of the solid is carried out by precipitation of the lignin upon acidification of the solution.

For that two possibilities are available:
1) Acidification and precipitation from the pulping solution without concentrating,
2) Acidification and precipitation from the pulping solution following concentration, e.g. by vaporization, (vacuum) distillation, ultra-filtration, nano-filtration.

In a further aspect in a method according to the present invention before concentrating the lignin in the alkaline, alcoholic solution by acid precipitation an additional concentration step by distillation, or vaporization, or vacuum vaporization, or ultra-filtration, or nano-filtration, or a combination of such steps, in particular by ultra-filtration, or nano-filtration is carried out.

In a further aspect in a method according to the present invention an alkaline, alcoholic solution of lignin is such a solution which has been prepared by preparing lignin from lignocellulose material via pulping with alcohol, in particular with a C₁₋₄ alcohol, in particular with ethanol, water and a base, in particular sodium hydroxide, at a temperature below 100° C., in particular from 40° C. to 90° C., in particular from 50° C. to 70° C., in particular in such a way that the concentration of the base, based on the dry weight of the lignocellulose to be pulped, is from 3 to 12% by weight, in particular from 4 to 10% by weight, in particular from 5 to 8% weight, and which has been concentrated, in particular by vaporization, (vacuum) distillation, ultra-filtration, nano-filtration.

In a method according to the present invention, 60-95%, preferably 70-95%, especially preferably 80-95%, of the dissolved lignin present in the pulping solution are maintained in the retentate by nano-filtration. The lignin concentrated in the retentate is separated by precipitation. E.g. by concentrating the lignin in the retentate by nano-filtration and the resulting volume reduction of the retentate, the amount of the acid required for precipitation is reduced by 50-90%, preferably by 70-90%, especially preferably by 80-90%. The permeate which is in an amount of the pulping solution of 60-90%, preferably of 70-90%, especially preferably of 80-90% and containing only residual amounts of lignin, is returned to the pulp. The permeate which is returned is subjected to a further purification step, in particular a reverse osmosis, distillation, precipitation of salts, ion exchange treatment, Variant 1) has the disadvantage that high concentrations of acids are necessary in order to decrease the alkaline pulp solution from about pH 11 to pH 7-pH 1. That is also true for variant 2 with concentrating by vaporization or (vacuum) distillation, since the concentration of the OH ions in the solution will not be modified thereby.

It has surprisingly been shown, however, that for concentrating an ultra- and/or nano-filtration is especially advantageous. That is because, depending on the extent of concentration, e.g. about 80% of the OH ions remain in the permeate and are returned into the pulping and only about 20% remain pH effective when concentrating the lignin by ultra- and/or nano-filtration.

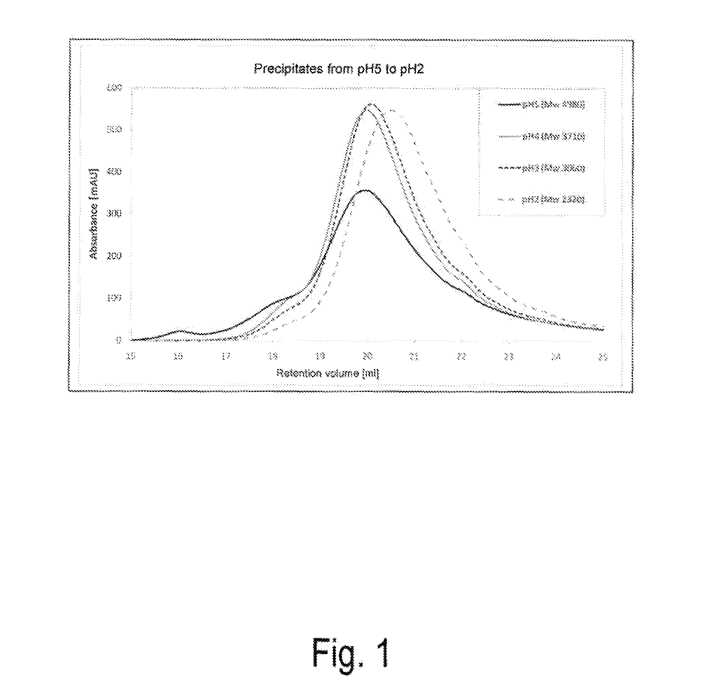

It has further been shown that with decreasing pH value also the molecular weight of the lignin traction prepared decreases. A typical molecular weight distribution is set out in the picture in FIG. 1.

The ultra- and/or nano-filtration for concentrating the lignin in an alkaline, alcoholic solution is novel and also subject of the present invention.

In a further aspect the present invention provides a method for the preparation of a concentrated, alkaline, alcoholic solution of lignin, characterized in that an alkaline, alcoholic solution of lignin, in particular such a solution that has been obtained according to one of the above described pulping methods, is subjected to a membrane filtration, such as an ultra- and/or nano-filtration, thereby optionally removing partially alcohol.

Optionally the permeate, before being returned into the pulping process, is subjected to a further purification step or several purification steps, in particular to reverse osmosis, distillation, precipitation of salts, ion exchange treatment.

If the concentration is carried out by membrane filtration and by partial removal of alcohol, these two steps may be carried out in any order; however, concentrating the lignin by membrane filtration with subsequent separation of the alcohol is preferred, because in the membrane filtration the solution is reduced in volume to about 20% and, hence, the (thermal) energy to be used for the separation of the alcohol will be reduced significantly. Furthermore, the returned solution already contains ethanol again for a further pulping step.

Concentrating the lignin by partial removal of the alcohol may only be carried out to such an extent that the low-molecular lignin thereby is maintained in solution. In the case of complete removal of the alcohol, a separation of the lignin into a low-molecular fraction remaining in solution upon acidification and a precipitating high-molecular fraction is not possible anymore, since the entire lignin precipitates.

The precipitation of the lignin from the retentate of the (ultra-)/nano filtration may be carried out by acidification of the lignin-containing solution in one step, or may be carried out in individual fractions, stepwise in different pH-value-steps from pH=7 to pH=1. This is true for a lignin solution containing alcohol as well as for a lignin solution, from which the alcohol has been removed.

For acidification in a method according to the present invention, an inorganic or an organic acid may be used. Preferably an inorganic acid, for example H₂SO₄, H₃PO₄, HCl is used.

The presence of alcohol in the lignin solution does have substantial influence on the precipitability of certain portions of the lignin fraction: if precipitation is carried out without alcohol removal in advance by vaporization or (vacuum) distillation, only about 50% of the lignin will be precipitated, with the other half remaining in solution.

The determination of the average molecular weight of the two fractions, which has been carried out as stated above, has shown that the precipitated fraction has an average Mn of 1150 D and an Mw of 5000 D, thus being high-molecular, while the soluble fraction is low-molecular with an average Mn of 750 D and an Mw of 1250.

The soluble fraction is precipitated, optionally fractionated, by removal of the alcohol;

The lignin prepared according to the method according to the invention or the individual lignin fractions, respectively, may be washed with suitable solvents, e.g. water or organic solvents, wherein the lignin fractions are not soluble, in order to separate contaminations such as salts, low-molecular compounds.

The lignin fractions prepared may be dried. The alcohol eventually still contained in the lignin fraction/s prepared may be recovered.

The lignin prepared according to the method according to the invention is preferably further processed into higher-value products, in particular phenol resins, fuel components, carbon fibres, plastic co-polymers or pharmaceutical products. Further processing may be carried out as appropriate, for example by well-known analogous methods for lignin processing. For example, the low-molecular lignin prepared may be processed into products requiring a low molecular weight, such as lignin derivates with a molecular weight of up to 1000, e.g. for the preparation of resins for the production of high-pressure laminates, like compact plates and laminates, and the higher-molecular lignin may be processed into products requiring a molecular weight of substantially more than 1000, such as for the preparation of resins which are used for the production of wood fibre boards.

Water-soluble substances present in the liquid supernatant of the lignin precipitations, in particular acids, acid-soluble lignin portions, salts, may be separated from the solution prepared in further steps upon isolation of the precipitated lignin. This approach is especially suitable for the separation of low-molecular hydroxy cinnamic acids, such as ferulic and coumaric acid, which may be separated by additional separation processes, e.g. adsorption to carrier materials or liquid/liquid extraction with appropriate solvents.

Another possibility is the use of PVPP in a packed column, through which the solution containing hydroxy cinnamic acids is moved. Thereby higher charges may be obtained and the ratio of PVPP used to the liquid amount to be purified is about 1:500 or higher.

In a further aspect the present invention provides a method according to the present invention, characterized in that, following the separation of the lignin, the remaining acid solution is treated with polyvinyl polypyrrolidone in order to isolate hydroxy cinnamic acids from the solution, which hydroxy cinnamic acids are adsorbed to the polyvinyl polypyrrolidone and removed from the solution.

The possibility of straightforward separation of the lignin fraction into a high- and a low-molecular fraction, each having their special ranges of use, is an essential advantage of a method according to the present invention.

1. A method for the preparation of lignin, characterized in that an acid is added to an alkaline, alcoholic solution of lignin and the precipitated lignin is separated, and, optionally, from the filtrate alcohol is removed in order to get further lignin.

2. A method according to claim 1, characterized in that an acid is added to an alkaline, alcoholic solution of lignin, the precipitated higher-molecular lignin fraction is separated and from the solution obtained upon separation alcohol is removed, in particular by vaporization, vacuum vaporization, distillation, vacuum distillation, in order to precipitate low-molecular lignin fractions.

3. A method according to claim 1 for preparing lignin from lignocellulosic material by pulping with alcohol, in particular a C₁₋₄ alcohol, in particular ethanol, water and a base, in particular sodium hydroxide, at a temperature below 100° C., in particular from 40° C. to 90° C., in particular from 50° C. to 70° C., in particular such, that the concentration of the base, based on the dry weight of the lignocellulose to be pulped, is from 3 to 12% by weight, in particular from 4 to 10% by weight, in particular from 5 to 8% by weight, characterized in that the lignin present in the alkaline, alcoholic solution is separated from the solid, in particular separated by pressing, centrifugation, filtration, and the lignin is separated from the pulping solution by addition of an acid in the form of a soluble, lower-molecular and an insoluble higher-molecular lignin fraction, in particular wherein the higher-molecular fraction is precipitated by addition of the acid and is subsequently separated, and the low-molecular fraction is precipitated by removal of the alcohol from the solution and is subsequently separated.

4. A method according to claim 1, wherein the lignocellulosic substrate is straw, in particular wheat straw.

5. A method according to claim 1, wherein the average molecular weight of the low-molecular lignin fraction has an Mn (number average molecular weight) of 1000 D to 650 D, in particular 750 D, and an Mw (weight average molecular weight) of 1500 D to 1000 D, in particular 1250 D; and the average molecular weight of the high-molecular lignin fraction has an Mn of 1500 D to 1000 D, in particular 1100 D to 1200 D, and an Mw of 7000 D to 3000 D, in particular 4500 D to 5000 D.

6. A method according to claim 1, wherein the pH of the alcoholic, alkaline solution, in particular pulping solution, is decreased by addition of an acid to a pH value from pH 7 to pH 1, in particular from pH 7 to pH 2, in particular- to (about) pH 2.

7. A method according to claim 1, wherein the pH value of the alcoholic, alkaline solution is decreased step-by-step by addition of an acid between pH 7 and pH 1, wherein the precipitated lignin is separated, in particular separated by centrifugation, filtration, ultra-filtration, following each step of the pH decrease, in particular wherein the lignin fractions obtained are dried and the alcohol possibly still present in the lignin fraction/s obtained is recovered by drying.

8. A method according to claim 1, wherein before concentrating the lignin in the alkaline, alcoholic solution by acid precipitation an additional concentration step by distillation, or vaporization, or vacuum vaporization, or ultra-filtration, or nano-filtration, or a combination of such steps, in particular by ultra-filtration, or nano-filtration, is carried out.

9. A method for the preparation of a concentrated, alkaline, alcoholic solution of lignin, characterized in that an alkaline, alcoholic solution of lignin, in particular a solution that has been obtained by a pulping method described in preceding claims, is subjected to a membrane filtration, in particular to an ultra- or nano-filtration, thereby optionally removing partially alcohol.

10. A method according to claim 1, wherein the low-molecular lignin obtained is processed into products requiring a lower molecular weight, in particular for the preparation of resins for the production of high-pressure laminates like compact laminates and laminates; and the higher-molecular lignin is processed into products requiring a higher molecular weight, in particular for the preparation of resins for the production of wood fibre boards.

11. A method according to claim 1, wherein following the separation of the lignin, the remaining acid solution is treated with polyvinyl polypyrrolidone in order to isolate hydroxy cinnamic acids from the solution, which hydroxy cinnamic acids are adsorbed to the polyvinyl polypyrrolidone and removed from the solution.
